(19) United States
(12) Patent Application Publication
(10) Pub. No.: US 2022/0266164 A1
(43) Pub. Date: Aug. 25, 2022

(54) SEPARATION OF CANNABINOIDS FROM CANNABINOID MIXTURES BY DERIVATIZATION

(71) Applicant: CANOPY GROWTH CORPORATION, Smiths Falls (CA)

(72) Inventors: Christopher ADAIR, Smiths Falls (CA); Ben GEILING, Smiths Falls (CA); Logan MARYNISSEN, Smiths Falls (CA)

(21) Appl. No.: 17/631,577

(22) PCT Filed: Jul. 30, 2020
(86) PCT No.: PCT/CA2020/051049

§ 371 Date: Jan. 31, 2022

Related U.S. Application Data
(60) Provisional application No. 62/881,027, filed on Jul. 31, 2019.

(51) Int. Cl. B01D 1/06; C07G 5/00; B01D 3/14

(52) U.S. Cl. CPC B01D 1/065; C07G 5/00; B01D 3/148

(57) ABSTRACT

The present disclosure relates to isolating one or more cannabinoids from an input mixture. There is disclosed an apparatus that comprises a first reaction vessel, a volatizing unit, and a distillation unit. The first reaction vessel provides a derivatized input mixture that comprises one or more derivatized cannabinoids. The volatizing unit volatilizes the derivatized input mixture into a derivatized cannabinoid-containing vapor-stream and a residue. The distillation unit receives the derivatized cannabinoid-containing vapor stream and separates a first derivatized cannabinoid within the derivatized cannabinoid-containing vapor stream from at least a second cannabinoid. There is also a method that comprises the steps of derivatizing one or more cannabinoids in an input mixture; volatilizing the derivatized input mixture to provided a derivatized cannabinoid-containing vapor stream; conducting the derivatized cannabinoid-containing vapor stream to distillation unit; and collecting a product that comprises the first derivatized cannabinoid.

### CROSS-REFERENCE TO RELATED APPLICATION

This application claims priority to and benefit of U.S. Provisional Patent Application Ser. No. 62/881,027 filed on Jul. 31, 2019, which is hereby incorporated by reference.

### TECHNICAL FIELD

The present disclosure generally relates to the isolation of cannabinoids and mixtures of cannabinoids. In particular, the present disclosure relates to separation of cannabinoids from cannabinoid-containing resins or concentrates to isolate pure compounds or purified mixtures.

### BACKGROUND

Naturally available cannabinoids, such as phytocannabinoids that are derived from plants, are typically sourced as a mixture of various cannabinoid compounds. Other plant components include, but are not limited to, lipids such as triglycerides and waxes. Due to the complexity of the mixture, it is challenging to fully understand the pharmacologic effect of an individual cannabinoid and mixtures thereof. Furthermore, isolating individual cannabinoids from a source mixture has also proven challenging. Known methods for separating cannabinoids from each other in a mixture of cannabinoids include chromatography, short path distillation, and crystallization.

In chromatography, an input material is dissolved in a solvent to form a mobile phase. The mobile phase passes through a chromatography column. When the mobile phase containing the dissolved mixture passes through the chromatography column, the compounds within the mobile phase can travel at different speeds. The amount of time it takes for a compound to travel through the column is called retention time and it is a result of size, shape, total charge, hydrophobic groups present on the compound's surface and binding capacity with the chromatography column. A widely used chromatography method for purification of molecules is High Pressure Liquid Chromatography (HPLC). This method involves passing the mobile phase through a chromatography column under pressures ranging from 10-400 atm and at a high flow rate. During HPLC, the use of small particles and the application of a high pressure increases the separation of the compounds within the mobile phase, which results in a shorter analysis time. However, HPLC requires complex and expensive equipment, generates significant waste, and is costly because of the large quantities of organic solvent required. Generally speaking, organic solvents present specific and further challenges because they can be expensive, toxic if consumed, and they are typically highly flammable and, therefore, they require specific equipment to handle safely. Other forms of chromatography, such as flash chromatography, simulated moving bed chromatography, and centrifugal partition chromatography can all suffer from similar challenges as HPLC with respect to complexity and solvent requirements.

Short path distillation involves a distillate travelling a short distance and it is typically performed under high vacuum to allow for the separation of larger heat-sensitive compounds. This method can run continuously and has the advantage of not using solvents to achieve the desired separation. However, short path distillation relies on a significant difference in vapor pressure between the compounds being separated. While this vapor pressure difference may hold true between cannabinoid compounds, as a whole, versus other components of plant-derived mixtures, the separation of individual cannabinoids from each other, or even from some waxes and oils, is not readily achievable by short path distillation.

Crystallization is another methodology that is known for separating cannabinoids from a mixture. Crystallization is possible with some cannabinoid compounds in the neutral form (primarily cannabigerol and cannabidiol) and the carboxylic acid form of all cannabinoid compounds. Crystallization is challenging because it often takes considerable time. Crystallization also uses organic solvent and it is typically only able to produce isolates/single compounds—not complex mixtures. Furthermore, crystallization is traditionally a batch style process, which limits its utility or even prevents its integration into a continuous processing method. Crystallization also requires a high purity input prior to beginning the process in order to maximize yields. This means that an alternate separation technology is typically used before crystallization, which results in an overall inefficiency in the separation methodology.

Each of the known methodologies for separating cannabinoids from a mixture are challenged by inefficient and incomplete separation of desired compounds from the mixture, high operational costs and, often times, the necessity of using organic solvents. However, given the potential pharmacological value of individual cannabinoids, improved separation methods to provide highly pure individual cannabinoids—or mixtures of cannabinoids with specific relative ratios of one compound to another compound—may be desirable.

### SUMMARY

The present disclosure relates to apparatuses and methods for isolating one or more cannabinoids from an input mixture.

In an embodiment, the present disclosure relates to an apparatus for isolating one or more cannabinoids from an input mixture, the apparatus comprising: (a) first reaction vessel that is configured to receive the input mixture and carry out a first derivatization reaction to provide a derivatized input mixture that comprises one or more derivatized cannabinoids; (b) a volatizing unit that is configured to receive and volatilize the derivatized input mixture into a derivatized cannabinoid-containing vapor stream and a residue; and (c) a distillation unit configured to receive the derivatized cannabinoid-containing vapor stream and to separate a first derivatized cannabinoid within the derivatized cannabinoid-containing vapor stream from at least a second cannabinoid.

In an embodiment, the present disclosure relates to a method of isolating one or more cannabinoids from an input mixture, the method comprising steps of: (a) derivatizing one or more cannabinoids in the input mixture to form a derivatized input mixture that comprises one or more derivatized cannabinoids; (b) volatilizing the derivatized input mixture to provide a derivatized cannabinoid-containing vapor stream and a residue; (c) conducting the derivatized cannabinoid-containing vapor stream to a distillation unit to separate a first derivatized cannabinoid within the derivatized cannabinoid-containing vapor stream from at least a second cannabinoid; and (d) collecting a product that comprises the first derivatized cannabinoid.

In an embodiment, the present disclosure relates to a method of isolating one or more cannabinoids from an input mixture, the method comprising steps of: (a) volatilizing the input mixture to provide a cannabinoid-containing vapor stream and a residue; (b) derivatizing one or more cannabinoids in the cannabinoid-containing vapor stream to form a derivatized cannabinoid-containing vapor stream that comprises one or more derivatized cannabinoids; (c) conducting the derivatized cannabinoid-containing vapor stream to a distillation unit to separate a first derivatized cannabinoid within the derivatized cannabinoid-containing vapor stream from at least a second cannabinoid; and (d) collecting a product that comprises the first derivatized cannabinoid.

In some embodiments, the cannabis concentrate is a cannabis resin.

In some embodiments, the distillation unit is a fractional distillation unit.

Without being bound by any particular theory, derivatizing a first cannabinoid may sufficiently change its boiling point, or other physical properties, which may aid in separation of the first cannabinoid from a mixture by distillation. The derivatization may be later reversed to regenerate the first cannabinoid. The reversible derivatization reaction may improve the yields of substantially pure cannabinoids from the input mixture (e.g. cannabis resin). Some embodiments of the present disclosure may also relate to derivatizing more than one cannabinoid, separating each of the more than one cannabinoids from a mixture, then reversing the derivatization reaction to produce substantially pure yields of each of the more than one cannabinoids from the mixture.

### BRIEF DESCRIPTION OF DRAWINGS

These and other features of the present disclosure will become more apparent in the following detailed description in which reference is made to the appended drawings. The appended drawings illustrate one or more embodiments of the present disclosure by way of example only and are not to be construed as limiting the scope of the present disclosure.

FIG. 1 shows a schematic representation of an apparatus, according to an embodiment of the present disclosure, which comprises a first reaction vessel, and a volatizing unit in fluid communication with a distillation unit.

FIG. 2 shows a schematic representation of an embodiment of the distillation unit shown in FIG. 1 wherein the unit includes devices to control temperature and pressure and a plenum that houses column packing materials.

FIG. 3 shows a schematic representation of another apparatus, according to an embodiment of the present disclosure that includes a preliminary treatment unit that is upstream of the volatizing unit.

FIG. 4 shows a schematic representation of a preliminary treatment, according to at least one embodiment of the present disclosure, for use with the apparatus shown in FIG. 3.

FIG. 5 shows two schematic representations of methods according to the present disclosure, wherein FIG. 5A shows the steps of a first method; and FIG. 5B shows the steps of a second method.

FIG. 6 shows a line graph that represents the Residue Product (RP) ratio of CBD:THC plotted over the various distillation temperatures for Example 5.

FIG. 7 shows a line graph that represents the net weight versus the temperature of the residue and the distillate for Example 6.

FIG. 8 shows data for Example 7, wherein FIG. 8A shows a line graph that represents the enrichment of CBD:THC over the four trials; and, FIG. 8B shows a line graph that represents the percentage of CBD (upper line) and THC (lower line) in the distillate product.

FIG. 9 shows a histogram that represents the distillate net output compared with the pressure within the distillation unit for Example 8.

FIG. 10 shows a histogram that represents the distillate/residue split as compared to the feed rate for Example 8.

FIG. 11 shows a line graph that represents the distillate/residue split as compared to the various temperatures for Example 9.

### DETAILED DESCRIPTION

Embodiments of the present disclosure relate to at least one apparatus and at least one method for separating one or more cannabinoids from an input mixture.

Some embodiments of the present disclosure relate to an apparatus that comprises a first reaction vessel, a volatizing unit, and a fractional distillation unit. Some embodiments of the present disclosure relate to a method that comprises the steps of derivatizing the input mixture, volatilizing the derivatized input mixture, conducting a derivatized cannabinoid-containing vapor stream to a distillation unit, and separating one cannabinoid from one or more other cannabinoids within the derivatized input mixture, and optionally regenerating at least some of the original cannabinoids. The embodiments of the present disclosure may be suitable for use on an industrial scale and may have the advantages of being continuously operated while substantially reducing or avoiding the use of organic solvents.

In one aspect, the present disclosure relates to an apparatus for isolating one or more cannabinoids from a cannabis concentrate.

In an embodiment, an apparatus of the present disclosure comprises: (a) first reaction vessel that is configured to receive the input mixture and carry out a first derivatization reaction to provide a derivatized input mixture that comprises one or more derivatized cannabinoids; (b) a volatizing unit that is configured to receive and volatilize the derivatized input mixture into a derivatized cannabinoid-containing vapor stream and a residue; and (c) a distillation unit configured to receive the derivatized cannabinoid-containing vapor stream and to separate a first derivatized cannabinoid within the derivatized cannabinoid-containing vapor stream from at least a second cannabinoid. The mixing vessel may be any vessel capable of containing the input mixture in order to carry out the derivatization reaction. The mixing vessel may be of any suitable shape or size, and may for example be of industrial scale or labscale. Non-limiting examples of mixing vessels include industrial grade mixers, mixing tanks, and mixing drums.

As used herein, the term “volatizing unit” refers to a unit or component that can volatilize or evaporate a substance. The volatizing unit can be of any suitable structure for volatilizing the input mixture (derivatized or not). In an embodiment, the volatizing unit may be an evaporator, a wiped film evaporator, a short path distillation unit, a rising-falling film evaporator, a pot still, a jacketed or heated vessel, a centrifugal evaporator, a centrifugal short path distillation, or any combination thereof. In some embodiments of the present disclosure, the volatizing unit is a wiped film evaporator, such as an LCI LabVap® Thin-Film Evaporator that includes a heated surface and a rotating wiping system that physically agitates the input mixture therein for heat and mass transfer of the one or more cannabinoids (derivatized or not) to a vapor state. In some embodiments, the volatizing unit is a VKL70-5 Shortpath Distillation System for causing a heat and mass transfer of the one or more cannabinoids to a vapor state.

Wiped film evaporation is a method used for separation of thermally-sensitive components. The method provides a short residence time and low evaporation temperature, which help to prevent degradation of one or more thermally sensitive target-components.

As used herein, the term “distillation unit” refers to a unit or component that can perform a physical separation of compounds comprised in the cannabinoid-containing vapor stream (derivatized or not) based on boiling point of the compounds. In an embodiment, the distillation unit comprise a distillation column. The distillation column may be of any suitable shape or size including, but not limited to, a vertical cylindrical column or a distillation tower. In an embodiment, the distillation unit is a fractional distillation unit. A fractional distillation unit separates a mixture into one or more component parts or fractions. In an embodiment, the fractional distillation unit comprises a temperature control unit and/or a pressure control unit. In this regard, in certain embodiments, the distillation unit can be configured to perform both evaporation and condensation.

In an embodiment, the distillation column defines a plenum that may house packing material, or not. In an embodiment, the plenum is configured to separate one or more derivatized cannabinoids from at least one other cannabinoid. In an embodiment, the plenum is configured to separate a first derivatized cannabinoid from at least a second cannabinoid, at least a second and third cannabinoid, at least a second, third or fourth cannabinoid, or at least an even greater number of cannabinoids. In an embodiment, the plenum is configured to separate one or more derivatized cannabinoids from their own non-derivatized counterparts. In an embodiment, the plenum is configured to separate multiple derivatized cannabinoids for non-derivatized cannabinoids. In an embodiment, plenum is configured to separate 1, 2, 3, 4, 5, 6, 7, 8, 9, 10 or more derivatized cannabinoids.

As used herein, the term “cannabinoid” refers to: (i) a chemical compound belonging to a class of secondary compounds commonly found in plants of genus cannabis; and/or (iii) one of a class of diverse chemical compounds that may act on cannabinoid receptors such as CB1 and CB2.

In select embodiments of the present disclosure, the cannabinoid is a compound found in a plant, e.g., a plant of genus cannabis, and is sometimes referred to as a phytocannabinoid. One of the most notable cannabinoids of the phytocannabinoids is tetrahydrocannabinol (THC), the primary psychoactive compound in cannabis. Cannabidiol (CBD) is another cannabinoid that is a major constituent of the phytocannabinoids. There are at least 113 different cannabinoids isolated from cannabis, exhibiting varied effects.

In select embodiments of the present disclosure, the cannabinoid is a compound found in a mammal, sometimes called an endocannabinoid.

In many cases, a cannabinoid can be identified because its chemical name will include the text string “*cannabi*”. However, there are a number of cannabinoids that do not use this nomenclature, such as for example those described herein.

As well, any and all isomeric, enantiomeric, or optically active derivatives are also encompassed. In particular, where appropriate, reference to a particular cannabinoid includes both the “A Form” and the “B Form”. For example, it is known that THCA has two isomers, THCA-A in which the carboxylic acid group is in the 1 position between the hydroxyl group and the carbon chain (A Form) and THCA-B in which the carboxylic acid group is in the 3 position following the carbon chain (B Form).

Examples of cannabinoids include, but are not limited to, Cannabigerolic Acid (CBGA), Cannabigerolic Acid monomethylether (CBGAM), Cannabigerol (CBG), Cannabigerol monomethylether (CBGM), Cannabigerovarinic Acid (CBGVA), Cannabigerovarin (CBGV), Cannabichromenic Acid (CBCA), Cannabichromene (CBC), Cannabichromevarinic Acid (CBCVA), Cannabichromevarin (CBCV), Cannabidiolic Acid (CBDA), Cannabidiol (CBD), Δ6-Cannabidiol (Δ6-CBD), Cannabidiol monomethylether (CBDM), Cannabidiol-C4 (CBD-C4), Cannabidivarinic Acid (CBDVA), Cannabidivarin (CBDV), Cannabidiorcol (CBD-C1), Tetrahydrocannabinolic acid A (THCA-A), Tetrahydrocannabinolic acid B (THCA-B), Tetrahydrocannabinol (THC or Δ9-THC), Δ8-tetrahydrocannabinol (Δ8-THC), trans-Δ10-tetrahydrocannabinol (trans-Δ10-THC), cis-Δ10-tetrahydrocannabinol (cis-Δ10-THC),Tetrahydrocannabinolic acid C4 (THCA-C4), Tetrahydrocannabinol C4 (THC-C4), Tetrahydrocannabivarinic acid (THCVA), Tetrahydrocannabivarin (THCV), Δ8-Tetrahydrocannabivarin (Δ8-THCV), Δ9-Tetrahydrocannabivarin (Δ9-THCV), Tetrahydrocannabiorcolic acid (THCA-C1), Tetrahydrocannabiorcol (THC-C1), Δ7-cis-iso-tetrahydrocannabivarin, Δ8-tetrahydrocannabinolic acid (Δ8-THCA), Δ9-tetrahydrocannabinolic acid (Δ9-THCA), Cannabicyclolic acid (CBLA), Cannabicyclol (CBL), Cannabicyclovarin (CBLV), Cannabielsoic acid A (CBEA-A), Cannabielsoic acid B (CBEA-B), Cannabielsoin (CBE), Cannabinolic acid (CBNA), Cannabinol (CBN), Cannabinol methylether (CBNM), Cannabinol-C4 (CBN-C4), Cannabivarin (CBV), Cannabino-C2 (CBN-C2), Cannabiorcol (CBN-C1), Cannabinodiol (CBND), Cannabinodivarin (CBDV), Cannabitriol (CBT), 11-hydroxy-Δ9-tetrahydrocannabinol (11-OH-THC), 11-nor 9-carboxy-Δ9-tetrahydrocannabinol, Ethoxy-cannabitriolvarin (CBTVE), 10-Ethoxy-9-hydroxy-Δ6a-tetrahydrocannabinol, Cannabitriolvarin (CBTV), 8,9 Dihydroxy-Δ6a(10a)-tetrahydrocannabinol (8,9-Di-OH-CBT-5), Dehydrocannabifuran (DCBF), Cannbifuran (CBF), Cannabichromanon (CBCN), Cannabicitran, 10-Oxo-Δ6a(10a)-tetrahydrocannabinol (OTHC), Δ9-cis-tetrahydrocannabinol (cis-THC), Cannabiripsol (CBR), 3,4,5,6-tetrahydro-7-hydroxy-alpha-alpha-2-trimethyl-9-n-propyl-2,6-methano-2H-1-benzoxocin-5-methanol (OH-iso-HHCV), Trihydroxy-delta-9-tetrahydrocannabinol (triOH-THC), Yangonin, Epigallocatechin gallate, Dodeca-2E, 4E, 8Z, 10Z-tetraenoic acid isobutylamide, hexahydrocannibinol, and Dodeca-2E,4E-dienoic acid isobutylamide.

As used herein, the term “THC” refers to tetrahydrocannabinol. “THC” is used interchangeably herein with “Δ9-THC”.

Structural formulae of cannabinoids of the present disclosure may include the following:

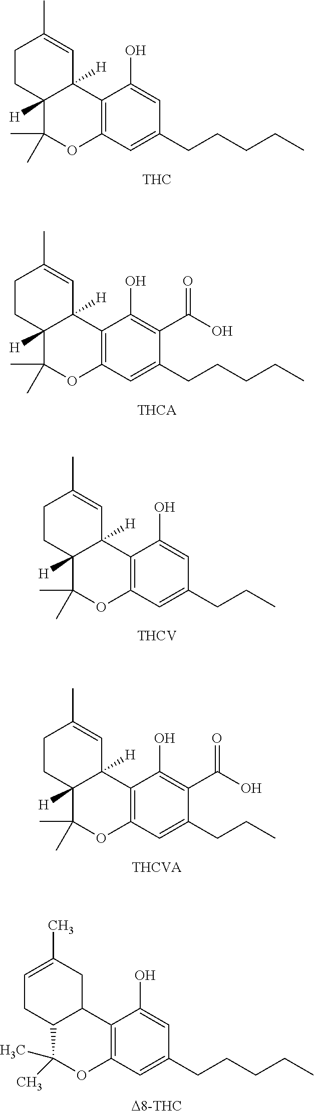

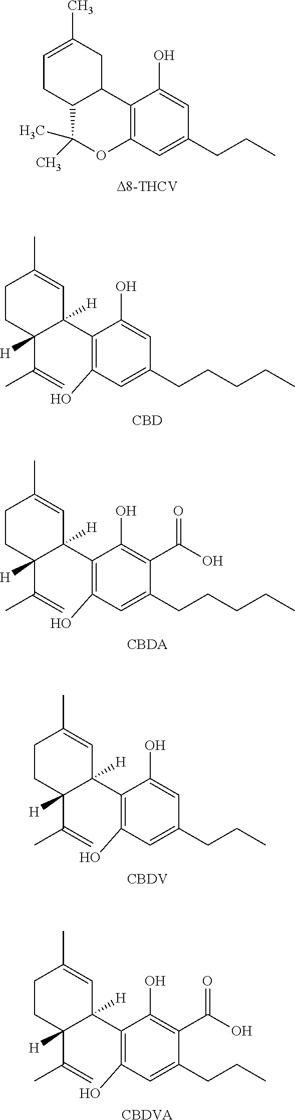

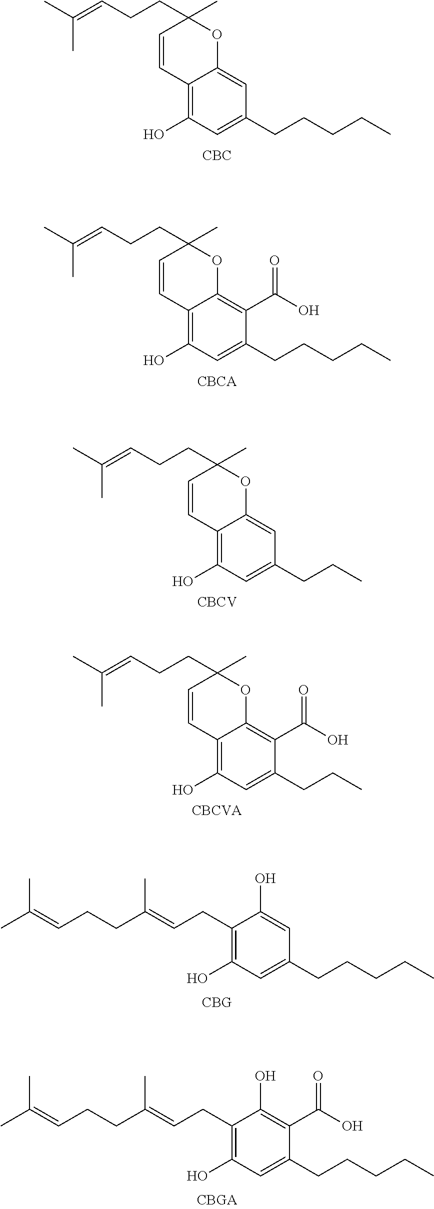

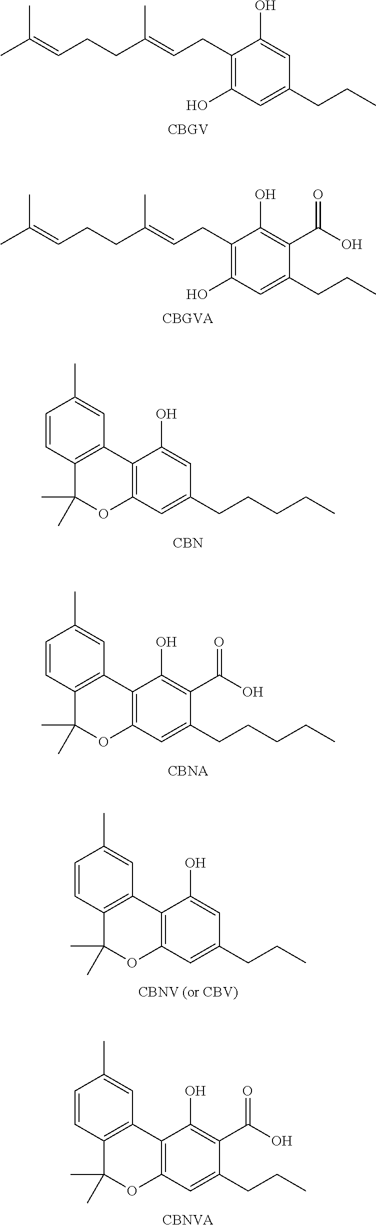

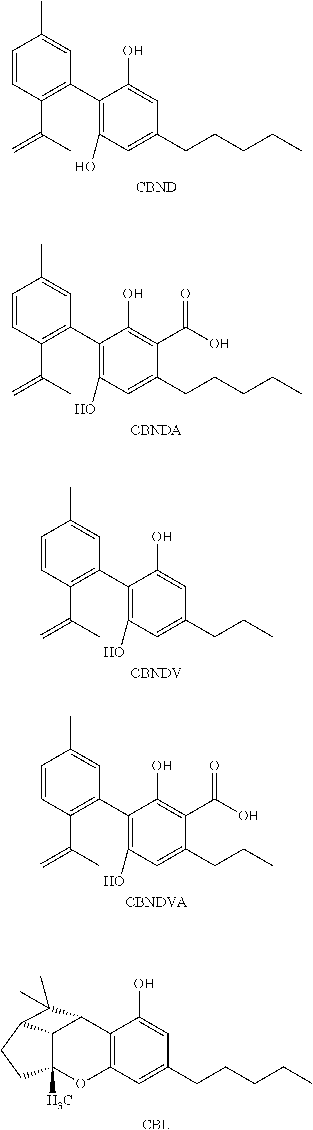

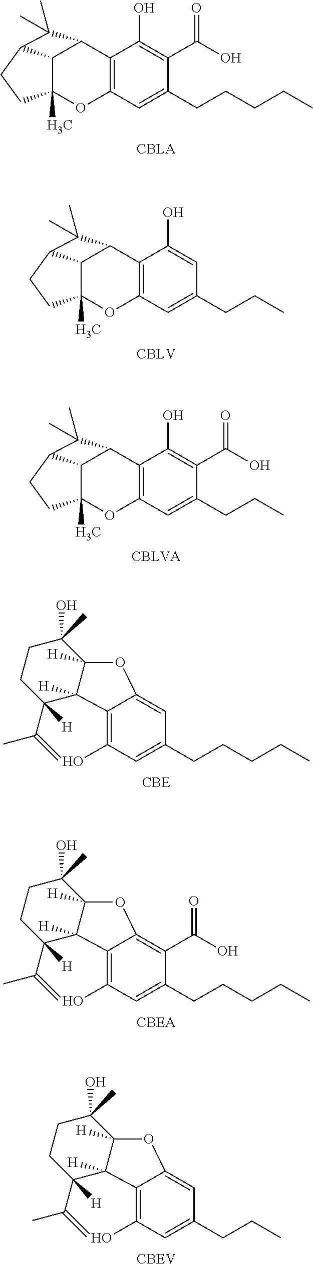

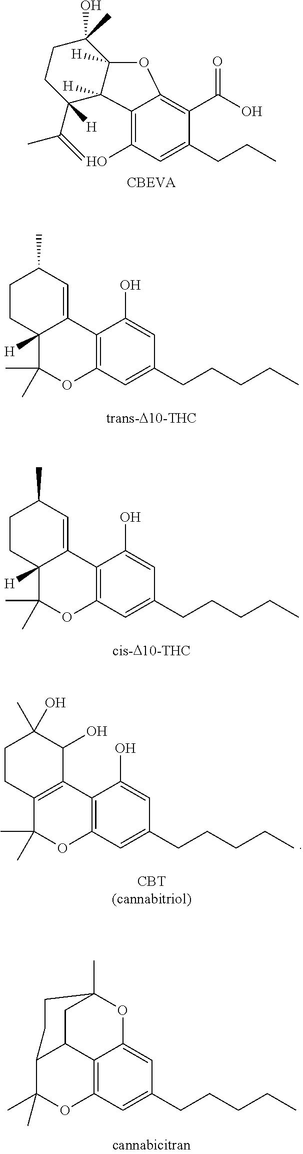

In the context of the present disclosure, the terms “first cannabinoid”, “second cannabinoid”, “third cannabinoid”, “fourth cannabinoid”, and so on, include and encompass any cannabinoid, including any of those described herein.

In select embodiments, the cannabinoid isolated by the apparatus and methods disclosed herein may for example and without limitation be any of those described herein. In a particular, embodiment, the cannabinoid isolated by the apparatus and methods disclosed herein may be THC, Δ8-THC, trans-Δ10-THC, cis-10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

In select embodiments of the present disclosure, the isolated cannabinoid may comprise CBD, CBDV, CBC, CBCV, CBG, CBGV, THC, THCV, or a regioisomer thereof. As used herein, the term “regioisomers” refers to compounds that differ only in the location of a particular functional group.

In an embodiment, the isolated cannabinoid is THC.

In an embodiment, the isolated cannabinoid is CBD.

As used herein, the term “input mixture” includes any cannabis plant material or extract thereof. Where the apparatuses or methods herein describe a “first input mixture” and a “second input mixture”, it is the first input mixture that is the original source material (i.e. plant material or extract thereof), whereas the subsequent input mixtures (e.g. second or third) are a mixture formed in accordance with the described methods using a first input mixture.

In an embodiment, the input mixture is a cannabis concentrate. As used herein, “cannabis concentrate” refers to a mixture of cannabinoids that is obtained from a cannabis plant, such as for example a mixture of compounds or compositions that have been extracted from cannabis. Non-limiting embodiments of a cannabis concentrate include a cannabis distillate, a cannabis isolate, a cannabis resin, or any other type of extract containing one or more cannabinoids or terpenes, or both. In an embodiment, the cannabis concentrate is a cannabis resin.

The apparatus and methods disclosed herein employ one or more derivatization reactions to provide a derivatized input mixture that comprises one or more derivatized cannabinoids. As used herein, by a “derivatization reaction” it is meant that a chemical modification is made to a compound (e.g. a cannabinoid) to provide a compound of different chemical structure (a derivative). In particular embodiments, the derivatization product (i.e. derivatized cannabinoid) will have one or more different chemical or physical properties than the original compound (e.g. such as a different vaporization temperature, boiling point, etc.).

In some embodiments of the present disclosure, the derivatization reaction may be reversible, e.g. a reversible chemical reaction. In such instances, the derivatized cannabinoid obtained by the derivatization reaction may be converted back to its original chemical structure by at least one chemical reaction. In an embodiment, the apparatus disclosed herein comprises one or more second reaction vessels in which at least one chemical reaction that reverses the derivatization reaction may be performed. The second reaction vessel may be of similar structure to the first reaction vessel.

In an embodiment of the apparatus and methods disclosed herein, one or more cannabinoids are derivatized before being volatilized and/or separated in a distillation unit. Such derivatization may sufficiently change their boiling point or other physical properties so as to advantageously aid in separation from other cannabinoids. As above, in certain embodiments, the derivatization maybe done in a manner that it is later reversible at high yield so that the original cannabinoid may be regenerated later.

In some embodiments, the derivatization reactions may be used to isolate individual cannabinoids (e.g. THC or CBD) or groups of cannabinoids.

Without limitation, exemplary embodiments of derivatization reactions include the formation of mono-alkyl ester cannabinoids; di-alkyl ester cannabinoids; a mono-aryl ester cannabinoids; di-aryl ester cannabinoids; monosulfonate cannabinoids; disulfonate cannabinoids; monosulfonic acid ester cannabinoids; disulfonic acid ester cannabinoids; mono-alkyl ether cannabinoids; di-alkyl ether cannabinoids; mono-alkyl silyl ether cannabinoids; di-alkyl silyl ether cannabinoids; or any combination thereof.

In some embodiments, the derivatization reaction incorporates a functional group on a base compound, in this case a cannabinoid, by a covalent bond. For example, an ester group may be added to a reactant cannabinoid, for example an acidic cannabinoid by an acid catalyzed reversible chemical reaction. Then an ester hydrolysis reaction can occur to produce the reactant cannabinoid.

Without limitation, any of the cannabinoids described herein may be derivatized within the apparatus and methods disclosed herein. For example, the derivatized cannabinoids may include, but are not limited to: mono-alkyl ester CBD; mono-alkyl ester CBG; mono-alkyl ester THC; mono-alkyl ester CBE; mono-alkyl ester CBN; mono-alkyl ester CBL; mono-alkyl ester THCV; mono-alkyl ester CBDV; di-alkyl ester CBD; di-alkyl ester CBG; di-alkyl ester THC; di-alkyl ester CBE; di-alkyl ester CBN; di-alkyl ester CBL; di-alkyl ester THCV; di-alkyl ester CBDV; mono-aryl ester CBD; mono-aryl ester CBG; mono-aryl ester THC; mono-aryl ester CBE; mono-aryl ester CBN; mono-aryl ester CBL; mono-aryl ester THCV; mono-aryl ester CBDV; di-aryl ester CBD; di-aryl ester CBG; di-aryl ester THC; di-aryl ester CBE; di-aryl ester CBN; di-aryl ester CBL; di-aryl ester THCV; di-aryl ester CBDV; monosulfonate CBD; monosulfonate CBG; monosulfonate THC; monosulfonate CBE; monosulfonate CBN; monosulfonate CBL; monosulfonate THCV; monosulfonate CBDV; disulfonate CBD; disulfonate CBG; disulfonate THC; disulfonate CBE; disulfonate CBN; disulfonate CBL; disulfonate THCV; disulfonate CBDV; monosulfonic acid ester CBD; monosulfonic acid ester CBG; monosulfonic acid ester THC; monosulfonic acid ester CBE; monosulfonic acid ester CBN; monosulfonic acid ester CBL; monosulfonic acid ester THCV; monosulfonic acid ester CBDV; disulfonic acid ester CBD; disulfonic acid ester CBG; disulfonic acid ester THC; disulfonic acid ester CBE; disulfonic acid ester CBN; disulfonic acid ester CBL; disulfonic acid ester THCV; disulfonic acid ester CBDV; mono-alkyl ether CBD; mono-alkyl ether CBG; mono-alkyl ether THC; mono-alkyl ether CBE; mono-alkyl ether CBN; mono-alkyl ether CBL; mono-alkyl ether THCV; mono-alkyl ether CBDV; di-alkyl ether CBD; di-alkyl ether CBG; di-alkyl ether THC; di-alkyl ether CBE; di-alkyl ether CBN; di-alkyl ether CBL; di-alkyl ether THCV; di-alkyl ether CBDV; mono-alkyl silyl ether CBD; mono-alkyl silyl ether CBG; mono-alkyl silyl ether THC; mono-alkyl silyl ether CBE; mono-alkyl silyl ether CBN; mono-alkyl silyl ether CBL; mono-alkyl silyl ether THCV; mono-alkyl silyl ether CBDV; di-alkyl silyl ether CBD; di-alkyl silyl ether CBG; di-alkyl silyl ether THC; di-alkyl silyl ether CBE; di-alkyl silyl ether CBN; di-alkyl silyl ether CBL; di-alkyl silyl ether THCV; or di-alkyl silyl ether CBDV.

As described elsewhere herein, in embodiments of the apparatus and methods disclosed herein, the derivatization reaction may be revisable such that the derivatized cannabinoid may be converted back and the original cannabinoid regenerated. Thus, the original cannabinoid may be isolated by the apparatus and methods disclosed herein.

By way of example and without limitation, in an embodiment:
i) the derivatized cannabinoid is a mono-alkyl ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
ii) thederivatized cannabinoid is a di-alkyl ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
iii) the derivatized cannabinoid is a mono-aryl ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
iv) the derivatized cannabinoid is a di-aryl ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
v) the derivatized cannabinoid is a monosulfonate ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
vi) the derivatized cannabinoid is a disulfonate ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
vii) the derivatized cannabinoid is a monosulfonic acid ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
viii) the derivatized cannabinoid is a disulfonic acid ester cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
ix) the derivatized cannabinoid is a mono-alkyl ether cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
x) the derivatized cannabinoid is a di-alkyl ether cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof;
xi) the derivatized cannabinoid is a monosilyl ether cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof; or
xii) the derivatized cannabinoid is a disilyl ether cannabinoid and the isolated cannabinoid is THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, cannabicitran, or a combination thereof.

In any of the reversible derivatization reactions herein, at least one chemical reaction may be used to convert the derivatized cannabinoid back to the original cannabinoid, including any one or more of the cannabinoids disclosed herein. For example, from a mono-alkyl ester cannabinoid, a di-alkyl ester cannabinoid, a mono-aryl ester cannabinoid, a di-aryl ester cannabinoid, a monosulfonate ester cannabinoid, a disulfonate ester cannabinoid, a monosulfonic acid ester cannabinoid, a disulfonic acid ester cannabinoid, a mono-alkyl ether cannabinoid, a di-alkyl ether cannabinoid, a monosilyl ether cannabinoid, or a disilyl ether cannabinoid, to THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

The apparatus as disclosed herein may advantageously be used to separate or isolate a first derivatized cannabinoid from a second (non-derivatized) cannabinoid. In further embodiments, the apparatus as disclosed herein may be used to separate or isolate a first derivatized cannabinoid from a second, a third, a fourth, or more cannabinoids.

In some embodiments, the apparatus as disclosed herein may be used to separate or isolate a first, second, third, fourth, or more derivatized cannabinoids from any number of other non-derivatized cannabinoids. In this manner, a purified mixture of cannabinoids may be prepared.

In some embodiments, the apparatus as disclosed herein may be used to separate or isolate one or more derivatized cannabinoids from one or more other derivatized cannabinoids.

In another aspect, the present disclosure relates to methods of isolating one or more cannabinoids from an input mixture (e.g. a cannabis resin)

In an embodiment, the present disclosure relates to a method of isolating one or more cannabinoids from an input mixture, the method comprising steps of: (a) derivatizing one or more cannabinoids in the input mixture to form a derivatized input mixture that comprises one or more derivatized cannabinoids; (b) volatilizing the derivatized input mixture to provide a derivatized cannabinoid-containing vapor stream and a residue; (c) conducting the derivatized cannabinoid-containing vapor stream to a distillation unit to separate a first derivatized cannabinoid within the derivatized cannabinoid-containing vapor stream from at least a second cannabinoid; and (d) collecting a product that comprises the first derivatized cannabinoid.

In an embodiment, the present disclosure relates to a method of isolating one or more cannabinoids from an input mixture, the method comprising steps of: (a) volatilizing the input mixture to provide a cannabinoid-containing vapor stream and a residue; (b) derivatizing one or more cannabinoids in the cannabinoid-containing vapor stream to form a derivatized cannabinoid-containing vapor stream that comprises one or more derivatized cannabinoids; (c) conducting the derivatized cannabinoid-containing vapor stream to a distillation unit to separate a first derivatized cannabinoid within the derivatized cannabinoid-containing vapor stream from at least a second cannabinoid; and (d) collecting a product that comprises the first derivatized cannabinoid.

The derivatizing may be performed by any suitable chemical reaction for providing a derivatized cannabinoid. In an embodiment, the derivatizing is by a single chemical reaction. In other embodiments, the derivatizing is by two or more chemical reactions. Non-limiting exemplary chemical reactions are described elsewhere herein, including reactions involving the formation of mono-alkyl ester cannabinoids; di-alkyl ester cannabinoids; a mono-aryl ester cannabinoids; di-aryl ester cannabinoids; monosulfonate cannabinoids; disulfonate cannabinoids; monosulfonic acid ester cannabinoids; disulfonic acid ester cannabinoids; mono-alkyl ether cannabinoids; di-alkyl ether cannabinoids; mono-alkyl silyl ether cannabinoids; di-alkyl silyl ether cannabinoids; or any combination thereof.

The volatilizing may be done by any suitable means to cause at least one cannabinoid in the input mixture to vaporize. In an embodiment, the volatilizing can be by an evaporator, a wiped film evaporator, a short path distillation unit, a rising-falling film evaporator, a pot still, a jacketed or heated vessel, a centrifugal evaporator, a centrifugal short path distillation or any combination thereof. In a particular embodiment, the volatilizing is by a wiped film evaporator as described elsewhere herein.

The conducting may be by any suitable means including, but not limited to, passing the input mixture through a conduit. Non-limiting examples of conduits include tubing and piping. The conduit may be of any suitable size and material.

The collecting may be by any suitable means to obtain all or a portion of the product containing the derivatized cannabinoid. In an embodiment, the collecting of the product that comprises a derivatized cannabinoid may be by a distillation tray. A distillation tray may facilitate condensation of one or more cannabinoids. In an embodiment, the collecting is by a suitable container, such as a round-bottom flask or collection flask.

In the context of the present disclosure, the quantity of a cannabinoid in a particular composition may be expressed in various fashions, such as but not limited to a percentage of the total weight of the particular composition or as a ratio that represents the relative quantity of a cannabinoid as compared to another compound within the particular composition. Additionally, the relative quantities of two cannabinoids (for example a first cannabinoid relative to a second cannabinoid) in a particular composition may be expressed as a ratio (for example first cannabinoid:second cannabinoid). The relative quantities of cannabinoid products in a mixture may be referred to with analogous ratios (e.g. second cannabinoid:third cannabinoid). Those skilled in the art will recognize that a variety of analytical methods may be used to determine such quantities and ratios, and the protocols required to implement any such method are within the purview of those skilled in the art. By way of non-limiting example, such ratios may be determined by diode-array-detector high pressure liquid chromatography, UV-detector high pressure liquid chromatography, nuclear magnetic resonance spectroscopy, mass spectroscopy, flame-ionization gas chromatography, gas chromatograph-mass spectroscopy, or any combination thereof.

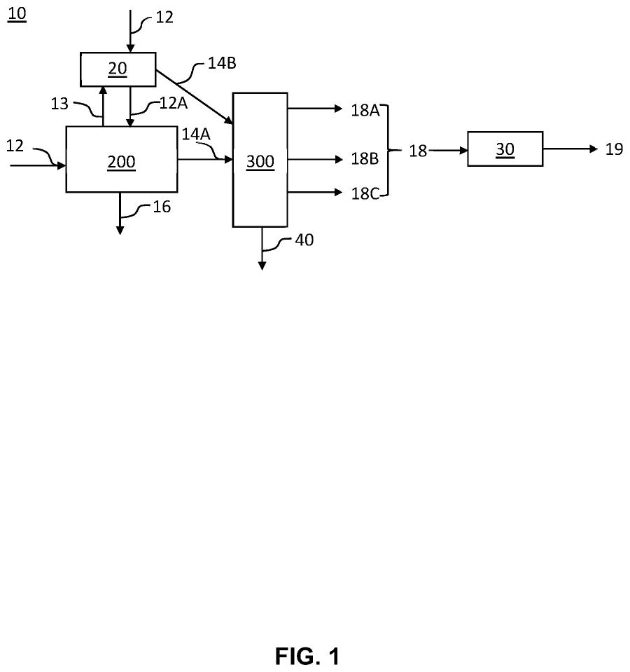

Embodiments of the present disclosure will now be described in detail including references to FIG. 1 to FIG. 5, which show embodiments of the apparatus and methods according to the present disclosure.

FIG. 1 shows a non-limiting example of an apparatus 10 that comprises a first reaction vessel 20, a volatizing unit 200, and a distillation unit 300 (which may also be referred to as a rectifier unit). In this embodiment, the first reaction vessel 20 may be configured to perform at least one derivatization reaction for making one or more derivatized cannabinoids from the cannabinoids that are constituents within the input mixture 12. In some embodiments of the present disclosure the at least one derivatization reaction is a “reversible chemical reaction”, such that the original cannabinoid can be regenerated by further chemical reaction at a later stage. As will be appreciated by those skilled in the art, there are a number of known reversible chemical reactions that can be performed at high enough yields to be commercially relevant. In some embodiments of the present disclosure the reversible chemical reaction can produce a derivatized cannabinoid that has a different physical property than the original (non-derivatized) cannabinoid. For example the derivatized cannabinoid may have a different boiling point than the original cannabinoid and that difference in boiling point may facilitate the separation of the derivatized cannabinoid from other cannabinoids within the input mixture 12, including the original cannabinoid itself or other derivatized cannabinoids.

In some embodiments, the derivatization reaction incorporates a functional group on a cannabinoid, such as by a covalent bond. For example, an ester group may be added to a reactant cannabinoid, for example an acidic cannabinoid, by an acid catalyzed reversible chemical reaction. Then an ester hydrolysis reaction can occur to produce the original (reactant) cannabinoid.

The input mixture 12 may be comprised of, but is not limited to, a mixture of cannabinoids, such as those cannabinoids described herein. Any of the cannabinoids herein may be a reactant cannabinoid in the derivatization reaction to produce a derivatized cannabinoid. For example, the derivatized cannabinoids may include, but are not limited to: mono-alkyl ester CBD; mono-alkyl ester CBG; mono-alkyl ester THC; mono-alkyl ester CBE; mono-alkyl ester CBN; mono-alkyl ester CBL; mono-alkyl ester THCV; mono-alkyl ester CBDV; di-alkyl ester CBD; di-alkyl ester CBG; di-alkyl ester THC; di-alkyl ester CBE; di-alkyl ester CBN; di-alkyl ester CBL; di-alkyl ester THCV; di-alkyl ester CBDV; mono-aryl ester CBD; mono-aryl ester CBG; mono-aryl ester THC; mono-aryl ester CBE; mono-aryl ester CBN; mono-aryl ester CBL; mono-aryl ester THCV; mono-aryl ester CBDV; di-aryl ester CBD; di-aryl ester CBG; di-aryl ester THC; di-aryl ester CBE; di-aryl ester CBN; di-aryl ester CBL; di-aryl ester THCV; di- aryl ester CBDV; monosulfonate CBD; monosulfonate CBG; monosulfonate THC; monosulfonate CBE; monosulfonate CBN; monosulfonate CBL; monosulfonate THCV; monosulfonate CBDV; disulfonate CBD; disulfonate CBG; disulfonate THC; disulfonate CBE; disulfonate CBN; disulfonate CBL; disulfonate THCV; disulfonate CBDV; monosulfonic acid ester CBD; monosulfonic acid ester CBG; monosulfonic acid ester THC; monosulfonic acid ester CBE; monosulfonic acid ester CBN; monosulfonic acid ester CBL; monosulfonic acid ester THCV; monosulfonic acid ester CBDV; disulfonic acid ester CBD; disulfonic acid ester CBG; disulfonic acid ester THC; disulfonic acid ester CBE; disulfonic acid ester CBN; disulfonic acid ester CBL; disulfonic acid ester THCV; disulfonic acid ester CBDV; mono-alkyl ether CBD; mono-alkyl ether CBG; mono-alkyl ether THC; mono-alkyl ether CBE; mono-alkyl ether CBN; mono-alkyl ether CBL; mono-alkyl ether THCV; mono-alkyl ether CBDV; di-alkyl ether CBD; di-alkyl ether CBG; di-alkyl ether THC; di-alkyl ether CBE; di-alkyl ether CBN; di-alkyl ether CBL; di-alkyl ether THCV; di-alkyl ether CBDV; mono-alkyl silyl ether CBD; mono-alkyl silyl ether CBG; mono-alkyl silyl ether THC; mono-alkyl silyl ether CBE; mono-alkyl silyl ether CBN; mono-alkyl silyl ether CBL; mono-alkyl silyl ether THCV; mono-alkyl silyl ether CBDV; di-alkyl silyl ether CBD; di-alkyl silyl ether CBG; di-alkyl silyl ether THC; di-alkyl silyl ether CBE; di-alkyl silyl ether CBN; di-alkyl silyl ether CBL; di-alkyl silyl ether THCV; or di-alkyl silyl ether CBDV.

In some embodiments of the present disclosure, the volatizing unit 200 may be any number of apparatus that can receive a derivatized input mixture 12A, which contains one or more derivatized cannabinoids. The volatizing unit 200 may be configured to volatilize at least a portion of the derivatized input mixture 12A to produce a derivatized cannabinoid-containing vapor-stream 14A and a residue 16 (e.g. non-volatized residue stream). For example, the volatizing unit 200 may apply heat, and optionally physical agitation, to the derivatized input mixture 12A for producing the derivatized cannabinoid-containing vapor-stream 14A and the residue 16. It will be appreciated by those skilled in the art that the derivatized cannabinoid-containing vapor stream 14A may comprise cannabinoids, one or more derivatized cannabinoids and, optionally, further constituents of the input mixture 12. In some embodiments of the present disclosure, substantially most, substantially all, or all of the cannabinoids, including the one or more derivatized cannabinoids, within the input mixture 12 are volatilized and entrained in the derivatized cannabinoid-containing vapor stream 14A. The residue 16 may be substantially free of cannabinoids, or not. In some embodiments, the residue 16 may be conducted back (not shown) to join either or both of the input mixture 12 and the derivatized input mixture 12A.

In some embodiments of the present disclosure, the volatizing unit 200 can be an evaporator, a wiped film evaporator, a short path distillation unit, a rising-falling film evaporator, a pot still, a jacketed or heated vessel, a centrifugal evaporator, a centrifugal short path distillation or any combination thereof. In some embodiments of the present disclosure, the volatizing unit 200 is a wiped film evaporator, such as an LCI LabVap® Thin-Film Evaporator that includes a heated surface and a rotating wiping system that physically agitates the derivatized input mixture 12A therein for heat and mass transfer of the one or more cannabinoids (derivatized or not) to a vapor state. In some embodiments of the present disclosure, the volatizing unit 200 is a VKL70-5 Shortpath Distillation System for causing a heat and mass transfer of the one or more cannabinoids to a vapor state.

In some embodiments, the derivatized cannabinoid-containing vapor stream 14A can be conducted, for example through one or more fluid conduits, to the distillation unit 300 for further processing, including at least separating one or more derivatized cannabinoids from other constituents of the derivatized cannabinoid-containing vapor stream 14A (e.g. other cannabinoids). The distillation unit 300 can be configured to separate heat sensitive compounds within the input mixture where such compounds have a relatively narrow boiling-point difference (FIG. 1). In some embodiments of the present disclosure, the distillation unit 300 may be a fractional distillation unit that separates at least a first derivatized cannabinoid from the remainder of the other cannabinoids (derivatized or not) and other constituents within the derivatized cannabinoid-containing vapor stream 14A. In some embodiments of the present disclosure, the distillation unit 300 may be a continuous fractional distillation unit that continuously separates at least the first derivatized cannabinoid from the remainder of the other cannabinoids (derivatized or not) and other constituents within the derivatized cannabinoid-containing vapor stream 14A. In some embodiments of the present disclosure, the distillation unit 300 can be configured to separate more than one derivatized cannabinoid from the other constituents within the derivatized cannabinoid-containing vapor stream 14A for producing at least one cannabinoid output stream 18, which may also be referred to as a distillate stream, and a waste stream 40. In some embodiments of the present disclosure, the waste stream 40 may be conducted back (not shown) to join one or more of the input mixture 12, the derivatized input mixture 12A or the derivatized cannabinoid-containing vapor stream 14A. While FIG. 1 shows three cannabinoid output streams 18A, 18B and 18C, the skilled person will appreciate that there may be more or less cannabinoid output streams produced by the distillation unit 300. The skilled person will also appreciate that the at least one cannabinoid output stream 18 may also comprise one or more output streams of a derivatized cannabinoid. The waste stream 40 may be collected from the second end 300B.

In some embodiments of the present disclosure, the apparatus 10 may further comprise a second reaction vessel 30. In these embodiments of the present disclosure, one or more of the cannabinoid output streams 18 can be conducted to the second reaction vessel 30, which can be configured to carry out at least one reverse derivatization reaction that regenerates at least a portion of the derivatized cannabinoids back into the original cannabinoid 19 (e.g. non-derivatized chemical state). For example, the at least one cannabinoid output stream 18 may actually be multiple output streams 18A, 18B, 18C (as the case may be) that each contain one or more derivatized cannabinoids. Each output stream may be conducted to an individual second reaction vessel 30 and the one or more derivatized cannabinoids may be converted back into the non-derivatized state that these compounds were in prior to entering into the first reaction vessel 20. These embodiments of the present disclosure may avoid mixing of the cannabinoids within each individual product stream.

Within the second reaction vessel 30, one or more of the following exemplary derivatized cannabinoids may be converted to non-derivatized cannabinoids, for example: mono-alkyl ester CBD may be converted to CBD; mono-alkyl ester CBG may be converted to CBG; mono-alkyl ester THC may be converted to THC; mono-alkyl ester CBE may be converted to CBE; mono-alkyl ester CBN may be converted to CBN; mono-alkyl ester CBL may be converted to CBL; mono-alkyl ester THCV may be converted to THCV; mono-alkyl ester CBDV may be converted to CBDV; di-alkyl ester CBD may be converted to CBD; di-alkyl ester CBG may be converted to CBG; di-alkyl ester THC may be converted to THC; di-alkyl ester CBE may be converted to CBE; di-alkyl ester CBN may be converted to CBN; di-alkyl ester CBL may be converted to CBL; di-alkyl ester THCV may be converted to THCV; di-alkyl ester CBDV may be converted to CBDV; mono-aryl ester CBD may be converted to CBD; mono-aryl ester CBG may be converted to CBG; mono-aryl ester THC may be converted to THC; mono-aryl ester CBE may be converted to CBE; mono-aryl ester CBN may be converted to CBN; mono-aryl ester CBL may be converted to CBL; mono-aryl ester THCV may be converted to THCV; mono-aryl ester CBDV may be converted to CBDV; di-aryl ester CBD may be converted to CBD; di-aryl ester CBG may be converted to CBG; di-aryl ester THC may be converted to THC; di-aryl ester CBE may be converted to CBE; di-aryl ester CBN may be converted to CBN; di-aryl ester CBL may be converted to CBL; di-aryl ester THCV may be converted to THCV; di-aryl ester CBDV may be converted to CBDV; monosulfonate CBD may be converted to CBD; monosulfonate CBG may be converted to CBG; monosulfonate THC may be converted to THC; monosulfonate CBE may be converted to CBE; monosulfonate CBN may be converted to CBN; monosulfonate CBL may be converted to CBL; monosulfonate THCV may be converted to THCV; monosulfonate CBDV may be converted to CBDV; disulfonate CBD may be converted to CBD; disulfonate CBG may be converted to CBG; disulfonate THC may be converted to THC; disulfonate CBE may be converted to CBE; disulfonate CBN may be converted to CBN; disulfonate CBL may be converted to CBL; disulfonate THCV may be converted to THCV; disulfonate CBDV may be converted to CBDV; monosulfonic acid ester CBD may be converted to CBD; monosulfonic acid ester CBG may be converted to CBG; monosulfonic acid ester THC may be converted to THC; monosulfonic acid ester CBE may be converted to CBE; monosulfonic acid ester CBN may be converted to CBN; monosulfonic acid ester CBL may be converted to CBL; monosulfonic acid ester THCV may be converted to THCV; monosulfonic acid ester CBDV may be converted to CBDV; disulfonic acid ester CBD may be converted to CBD; disulfonic acid ester CBG may be converted to CBG; disulfonic acid ester THC may be converted to THC; disulfonic acid ester CBE may be converted to CBE; disulfonic acid ester CBN may be converted to CBN; disulfonic acid ester CBL may be converted to CBL; disulfonic acid ester THCV may be converted to THCV; disulfonic acid ester CBDV may be converted to CBDV; mono-alkyl ether CBD may be converted to CBD; mono-alkyl ether CBG may be converted to CBG; mono-alkyl ether THC may be converted to THC; mono-alkyl ether CBE may be converted to CBE; mono-alkyl ether CBN may be converted to CBN; mono-alkyl ether CBL may be converted to CBL; mono-alkyl ether THCV may be converted to THCV; mono-alkyl ether CBDV may be converted to CBDV; di-alkyl ether CBD may be converted to CBD; di-alkyl ether CBG may be converted to CBG; di-alkyl ether THC may be converted to THC; di-alkyl ether CBE may be converted to CBE; di-alkyl ether CBN may be converted to CBN; di-alkyl ether CBL may be converted to CBL; di-alkyl ether THCV may be converted to THCV; di-alkyl ether CBDV may be converted to CBDV; mono-alkyl silyl ether CBD may be converted to CBD; mono-alkyl silyl ether CBG may be converted to CBG; mono-alkyl silyl ether THC may be converted to THC; mono-alkyl silyl ether CBE may be converted to CBE; mono-alkyl silyl ether CBN may be converted to CBN; mono-alkyl silyl ether CBL may be converted to CBL; mono-alkyl silyl ether THCV may be converted to THCV; mono-alkyl silyl ether CBDV may be converted to CBDV; di-alkyl silyl ether CBD may be converted to CBD; di-alkyl silyl ether CBG may be converted to CBG; di-alkyl silyl ether THC may be converted to THC; di-alkyl silyl ether CBE may be converted to CBE; di-alkyl silyl ether CBN may be converted to CBN; di-alkyl silyl ether CBL may be converted to CBL; di-alkyl silyl ether THCV may be converted to THCV; or di-alkyl silyl ether CBDV may be converted to CBDV.

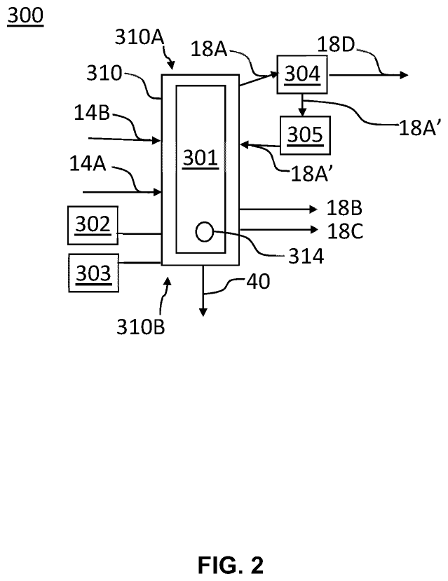

As shown in FIG. 2, the distillation unit 300 may comprise a distillation column 310 with a first end 310A, which may also be referred to as the column head, and a second end 310B. Together these two ends 310A, 310B define an internal plenum. The plenum 301 houses one or more separation trays (not shown) that may be configured to separate at least one derivatized cannabinoid from within the derivatized cannabinoid-containing vapor stream 14A from other constituents of the derivatized cannabinoid-containing vapour stream, which may include at least a second cannabinoid, a third cannabinoid, a fourth cannabinoid and further cannabinoids. As will be appreciated by the skilled reader, the one or more derivatized cannabinoids may be separated from each other, the non-derivatized cannabinoids and other constituents within the distillation unit 300 input feed.

In some embodiments, the distillation unit 300 may comprise both a temperature control unit 302 and pressure control unit 303. The temperature control unit 302 can be configured to heat or cool the temperature within the plenum 301. In some embodiments of the present disclosure the column 310 may be sealed at each end so that the plenum is isolated from the ambient pressure and the pressure control unit 303 is configured to increase or decrease the pressure within the plenum 301. In some embodiments of the present disclosure, the continuous fractional distillation unit 300 operates at a temperature of between about 100° C. and about 275° C. or between about 120° C. and about 150° C. within a range of pressures of between about 0.001 mbar and 110 mbar.

In some embodiments, the plenum 301 may house a packing material 314. The packing material 314 may include, but is not limited to, at least one of the following: MONTZ-Pak Type A3-750, MONTZ-Pak Type A3-1000, MONTZ-Pak Type A3-1200, MONTZ-Pak Type A3-1500, MONTZ-Pak Type A3-1900, Sulzer laboratory packing DX or any combination thereof.

In some embodiments, the distillation unit 300 may further comprise a condenser 304 that is operatively connected at, near, or to the first end 310A (see FIG. 2). The condenser 304 may be configured to condense a lighter vaporized cannabinoid fraction (for example the first cannabinoid output stream 18A, which may be the most volatile cannabinoid within the mixture of derivatized and non-derivatized cannabinoids) to form a condensed first cannabinoid output stream 18D. Optionally, a portion of the condensed distillate 18A can be conducted from the condenser 304 to one or more distributors 305. The distributors 305 may be configured to conduct a portion of the condensed steam 18A′ back into the plenum 301 where the portion of the condensed stream 18A′ is again mixed with the derivatized cannabinoid-containing vapor stream 14 in the distillation unit 300. Product streams 18B and 18C can be collected from the distillation unit 300.

In some embodiments of the present disclosure, some or all of the product of the first reaction vessel 20, which includes cannabinoids and at least one derivatized cannabinoid, may be directed via a fluid conduit 14B to the distillation unit 300 rather than to the volatizing unit 200.

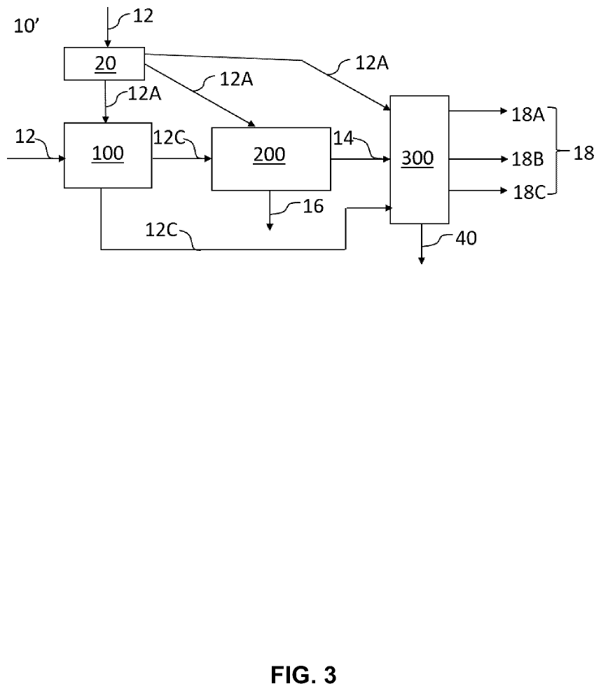

As shown in the non-limiting example of FIG. 3, some embodiments of the present disclosure relate to an apparatus 10′ that is configured to process the input mixture 12, the derivatized input mixture 12A or a combination thereof for producing a second input mixture 12C. The apparatus 10′ has many of the same components as the apparatus 10, with at least one difference being the processing unit 100. The second input mixture 12C (which comprises one or more derivatized cannabinoids from derivatized input mixture 12A) may then be conducted to the volatizing unit 200, the distillation unit 300 or a combination thereof. In some embodiments of the present disclosure, the derivatized input 12A may be conducted directly to the processing unit 100, to the volatizing unit 200, to the distillation unit 300 or any combination thereof. Those skilled in the art will appreciate that input mixture 12 may be introduced to processing unit 100, or not.

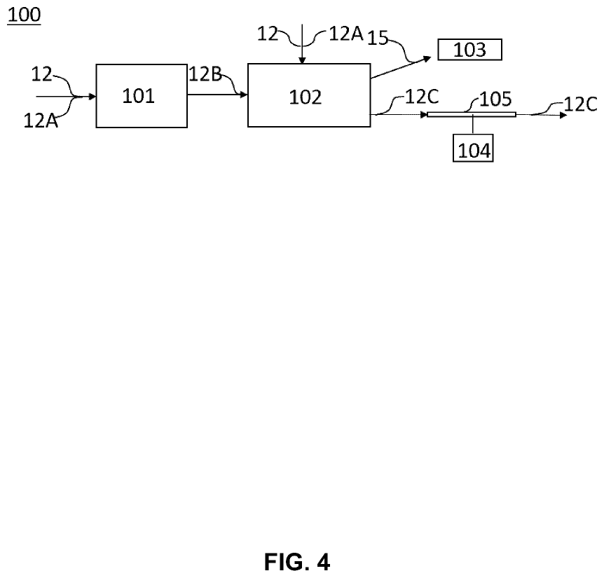

As shown in the non-limiting example of FIG. 4, some embodiments of the present disclosure relate to the processing unit 100 comprising a heatable tank 101, a degassing chamber 102 and a condenser 103. The heatable tank 101 is configured to receive the derivatized input mixture 12A or combinations of input mixture 12 and the derivatized input mixture 12A. In some embodiments of the present disclosure, the heatable tank 101 may be heated and, optionally, under vacuum for producing a heated input mixture 12B. The heated input mixture 12B (comprising one or more derivatized cannabinoids of derivatized input mixture 12A) may be conducted to the degassing chamber 102 that may also be operated under vacuum. Those skilled in the art will appreciate that the derivatized input mixture 12A or combinations of input mixture 12 and the derivatized input mixture 12A, may also be introduced to degassing chamber 102, or not. In some embodiments of the present disclosure, the condenser 103 may be used to condense one or more gaseous compounds 15 that are removed by the degassing chamber 102. The one or more gaseous compounds 13 can be condensable or non-condensable gases. Some examples of condensable gases include, but are not limited to: solvent residue (ethanol, butane and the like), volatile organic compounds, such as terpenes and water. The primary non-condensable gas is CO2. The processing unit 100 may also comprise a transfer pump 104 and a heated hose 105 that are configured to conduct the third input mixture 12C to either to the first end 310A of the column 310, the middle of the column 310, the volatizing unit 200, or any combination thereof.

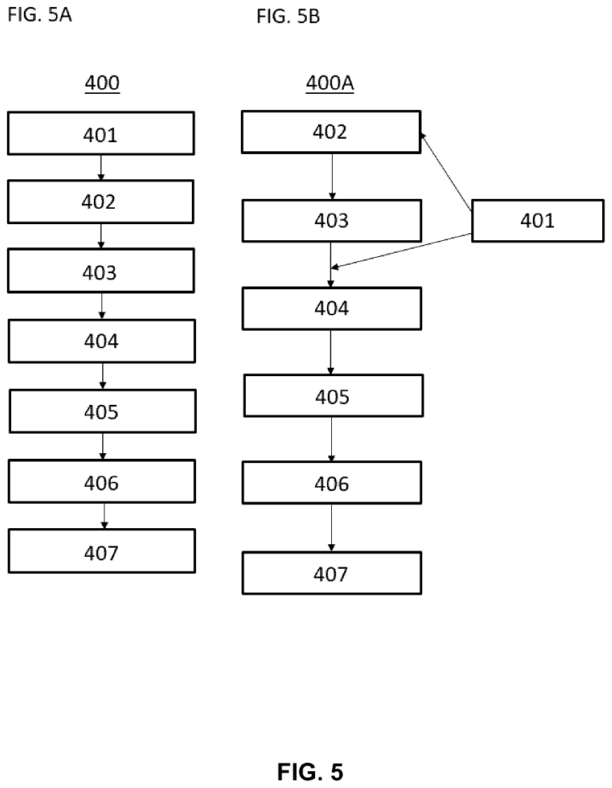

FIG. 5A shows a sequence of steps that form part of an embodiment of a first method 400 of the present disclosure that relates to separating cannabinoids from an input mixture by derivatization and fractional distillation.

The first method 400 comprises a step 401 of derivatizing the input mixture 12 and generating the derivatized input mixture 12A. As described herein above, the derivatized input mixture 12A may comprise one or more cannabinoids, one or more derivatized cannabinoids and other constituents of the input mixture 12.

Step 402 comprises a step of volatizing the derivatized input mixture 12A for example by heating and, optionally, physically agitating the derivatized input mixture 12A. As will be appreciated by those skilled in the art, the step 402 of volatizing may comprise a step of introducing the derivatized input mixture 12A into an apparatus for volatilizing the input mixture, for example the volatizing unit 200 described herein above.

Step 403 comprises separating the derivatized input mixture 12A from the residue stream 16 for generating the derivatized cannabinoid-containing vapor stream 14.

The method 400 further comprises a step 404 of conducting the derivatized cannabinoid-containing vapor stream 14 to a distillation unit and a step 405 of conducting the derivatized cannabinoid-containing vapor stream through the distillation unit. In some embodiments of the present disclosure, the distillation unit may be a fractional distillation unit that operates continuously or discontinuously. The fractional distillation unit may be operated at specific temperatures and pressures while performing a step 406 of isolating a first derivatized cannabinoid from at least a second cannabinoid within the fractional distillation unit.

The method 400 further comprises a step 407 of regenerating at least a portion of the original cannabinoids by reversing at least one derivatization reaction.

FIG. 5B shows another method 400A that includes many of the same steps as method 400 described above. At least one difference between method 400 and method 400A is that the step 401 of derivatizing one or more cannabinoids can occur before or after the step 403.

### EXAMPLE

### Example 1

### Derivatization

The input mixture containing cannabinoids may be a cannabis concentrate, such as cannabis resin, that is prepared by first preparing a precursor extract by the steps of dissolution, chilling and filtering to produce the precursor extract, which may be substantially transparent. Optionally, the precursor extract can be substantially or completely depleted of lipids and waxes. The precursor extract can have a cannabinoid concentration of between about 60% and about 90% (wt/wt) or between about 70% and about 80% (wt/wt).

The input mixture 12 can be derivatized with one or more functional groups including, but not limited to: mono-alkyl esters; di-alkyl esters; mono-aryl esters; di-aryl esters; monosulfonates; disulfonates; monosulfonic acid esters; disulfonic acid esters; mono-alkyl ethers; di-alkyl ethers; mono-alkyl silyl ethers; di-alkyl silyl ethers; or any combination thereof.

The derivatized input mixture 12A can be introduced into the volatizing unit 200. In some embodiments, the volatizing unit is a wiped film evaporator. The derivatized cannabinoids can evaporate from the mixture into derivatized cannabinoid-containing vapor stream 14A inside the volatizing unit 200 and can be conducted in the vapor phase to the continuous distillation unit 300. Non-volatile components 16 may be discharged from the bottom of the volatizing unit 200.

### Example 2

### Separation

The derivatized cannabinoid-containing vapor stream can be conducted through the fractional distillation column 310 where one or more derivatized cannabinoids in the derivatized cannabinoid-containing vapor stream may be separated from the other constituents of the vapor stream (e.g. other cannabinoids). The product stream 18 can be collected as a product from the distillation unit 300 and non-volatile components 40 are discharged from the bottom of the distillation unit 300.

### Example 3

### Regeneration

The product stream 18 collected from the distillation unit 300 may be transferred to the second reaction vessel 30, which may be configured to carry out one or more reactions to reverse at least one derivatization reaction and regenerate at least a portion of the original cannabinoids.

### Example 4

### Shortpath Distillation

In instances where the input mixture comprises volatile organic compounds (VOCs) and/or acidic cannabinoids, the input mixture may not be suitable for direct input into a short path distillation unit as the volatizing unit. This is because the VOCs can off gas and interfere with the vacuum pressure of the short path distillation unit. Additionally, the acidic cannabinoids can decarboxylate at the distillation temperatures contemplated herein, which generates CO₂ that can also interfere with the vacuum pressure of the short path distillation unit. Instead, this type of input mixture can be subjected to a two-staged process that includes a first degassing step and a second short path distillation step and then subjected to the fractional distillation process.

The first step includes degassing by heating these types of input mixtures to about 100° C. in a feed hopper and passing the heated input mixture through a 0.25 cubic foot horizontal wiped-film evaporator that is operating at a temperature of about 150° C. and at feed speed of about 150 mL per minute. The vacuum is maintained at about 0.5 atm absolute with appropriately sized condenser set to about 4° C. to collect any volatile compounds that are evaporated. This process effectively strips the input mixture of some or substantially all volatile compounds or acidic cannabinoids that may interfere later with shortpath distillation. Alternatively, the input mixture may be heated on a hotplate to about 150° C. with stirring until no more gas is produced.

After this degassing, the input mixture is maintained at about 100° C. and pumped into the shortpath distillation system where it is heated to between about 120° C. and about 250° C.—depending on the operating pressure—causing the cannabinoids to evaporate. The evaporated cannabinoids can be directed to the fractional distillation unit or they may be recondensed on an internal condenser set at about 50° C. and about 60° C. and any remaining low boiling point compounds are collected using a cold trap that is at about —40° C. ahead of the vacuum pump.

### Example 5

### Short Path Distillation for Enrichment of CBD at Various Temperatures

An input mixture was prepared using a plant input that had on average 14 wt/wt % CBD and <1 wt/wt % THC. The input mixture was subjected to a two-step shortpath distillation process (using the VKL70-5 Shortpath distillation system), as described herein above and then subjected to a distillation process at temperatures ranging between about 129° C. and 169° C. The cannabinoid concentration for the residue product (RP) and the distillation product (DP) are provided below in Table 1.

TABLE 1
Cannabinoid concentration of short path distilled
and further distilled input mixture.

| | Cannabinoid Concentration | | | | | | |
|---|---|---|---|---|---|---|---|
| | | | | | | | Residue |
| Temperature | RP | RP | RP Total | DP | DP | DP Total | Ratio: |
| (° C.) | CBD | THC | Cannabinoids | CBD | THC | Cannabinoids | CBD/THC |
| 169 | 3.39 | 0.38 | 3.77 | 67.72 | 2.67 | 75.15 | 8.92 |
| 164 | 2.53 | 2.49 | 82.15 | 82.15 | 2.95 | 89.87 | 14.32 |
| 159 | 3.64 | 3.64 | 81.33 | 81.33 | 2.58 | 88.56 | 12.74 |
| 154 | 4.52 | 4.52 | 82.27 | 82.27 | 2.42 | 88.53 | 13.76 |
| 149 | 3.06 | 3.06 | 83.28 | 83.28 | 2.28 | 89.48 | 27.80 |
| 144 | 3.21 | 3.21 | 88.33 | 88.33 | 2.03 | 94.42 | 28.45 |
| 139 | 2.59 | 2.59 | 82.81 | 82.81 | 1.41 | 87.5 | 33.97 |
| 134 | 1.75 | 1.75 | 81.87 | 81.87 | 0.95 | 85.8 | 49.33 |
| 129 | 1.33 | 1.33 | 78.62 | 78.62 | 0.92 | 82.58 | 68.60 |

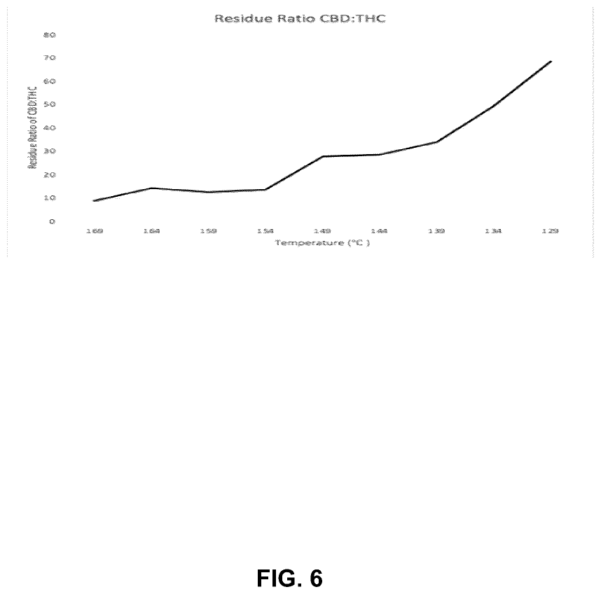

FIG. 6 shows a line graph that represents the RP ratio of CBD:THC plotted over the various distillation temperatures. Without being bound by any particular theory, THC can be distilled out to produce a higher concentration of CBD in a RP as shown by an increasing the ratio of CBD:THC to about 8× when temperatures are at about 129° C. as compared to higher temperatures.

### Example 6

### Short Path Distillation for Enrichment of CBD at Various Pressures

In this example, a second pass distillate was passed through a short path distillation unit (as described above in Example 6) and then subjected to the fractional distillation process at various pressures (2.6-3.0 mbar) and various temperatures (about 169° C. to about 234° C.) in order to assess the evaporation points and the purity of the resulting distillate and residue. Table 2 summarizes the residue/distillate accumulation over the various temperatures utilized.

TABLE 2
Residue and Distillate Accumulation Data

| | Residue/Distillate Accumulation | | | |
|---|---|---|---|---|
| | | | | Distillate/ |
| Temperature | Input | Residue | Distillate | Residue |
| (° C.) | Weight (g) | Weight (g) | Weight (g) | Split |
| 169 | 269.4 | 230.0 | 0 | 0 |
| 179 | 230.0 | 218.1 | 5.0 | 0.02 |
| 189 | 222.5 | 199.3 | 22.8 | 0.11 |
| 194 | 218.8 | 189.3 | 27.9 | 0.15 |
| 199 | 216.0 | 176.1 | 41.8 | 0.26 |
| 204 | 219.6 | 166.6 | 47.3 | 0.28 |
| 209 | 213.9 | 149.3 | 67.1 | 0.44 |
| 214 | 216.3 | 106.6 | 107.8 | 1.01 |
| 219 | 211.8 | 97.5 | 116.5 | 1.19 |
| 224 | 210.6 | 47.1 | 162.9 | 3.45 |
| 229* | 206.2 | 50.8 | 153.1 | 3.01 |
| 229 | 202.8 | 22.4 | 143.7 | 6.42 |
| 234* | 162.4 | 45.6 | 81.5 | 1.79 |
| 234 | 123.4 | 7.7 | 105.0 | 13.63 |

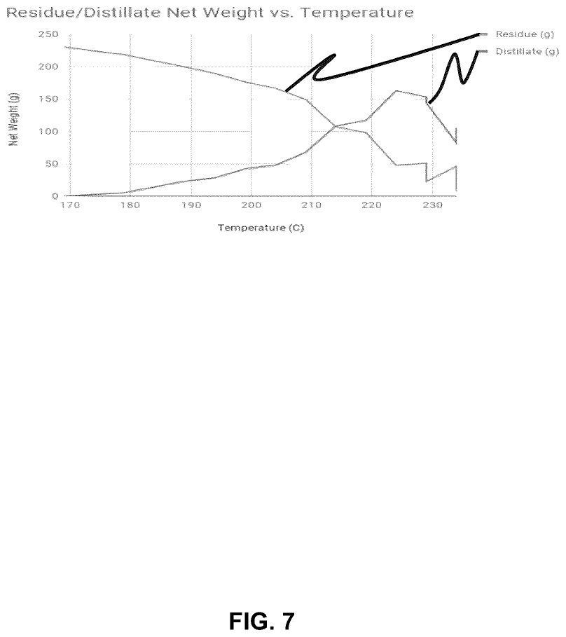

FIG. 7 shows a line graph that represents the net weight versus the temperature of the residue and the distillate, respectively.

Without being bound by any particular theory, the ratio of distillate to residue increased dramatically at about 214° C. This suggests that this may represent an optimal temperature point for evaporation of cannabinoids at 2.6-3 mbar. Higher temperatures were seen to cause burning of material on the inside of the distillation chamber.

### Example 7

### Constant Temperature and Constant Feed Rate

An input mixture was subjected to similar experimental conditions as described above in Example 6, but the distillation process was conducted at a constant temperature of 134° C. at a feed rate of 15 Hz. Table 3 summarizes the cannabinoid concentration of the RP and the DP.

TABLE 3
Cannabinoid Concentration of Residue Product and Distillate Product.

| | | Cannabinoid Concentration | | | | | | | |
|---|---|---|---|---|---|---|---|---|---|
| Trial | Temperature | RP | RP | RP Total | DP | DP | DP Total | Ratio: | Enrich- |
| # | (° C.) | CBD | THC | Cannabinoids | CBD | THC | Cannabinoids | CBD/THC | ment |
| 1 | 134 | 28.43 | 23.70 | 57.61 | 50.01 | 20.97 | 75.94 | 2.38 | 1.41 |
| 2 | 134 | 44.84 | 31.71 | 82.61 | 57.63 | 18.19 | 80.20 | 3.16 | 1.33 |
| 3 | 134 | 53.14 | 19.79 | 87.33 | 65.87 | 16.68 | 86.56 | 3.95 | 1.25 |
| 4 | 134 | 62.56 | 24.35 | 91.76 | 71.44 | 14.55 | 89.63 | 4.91 | 1.24 |

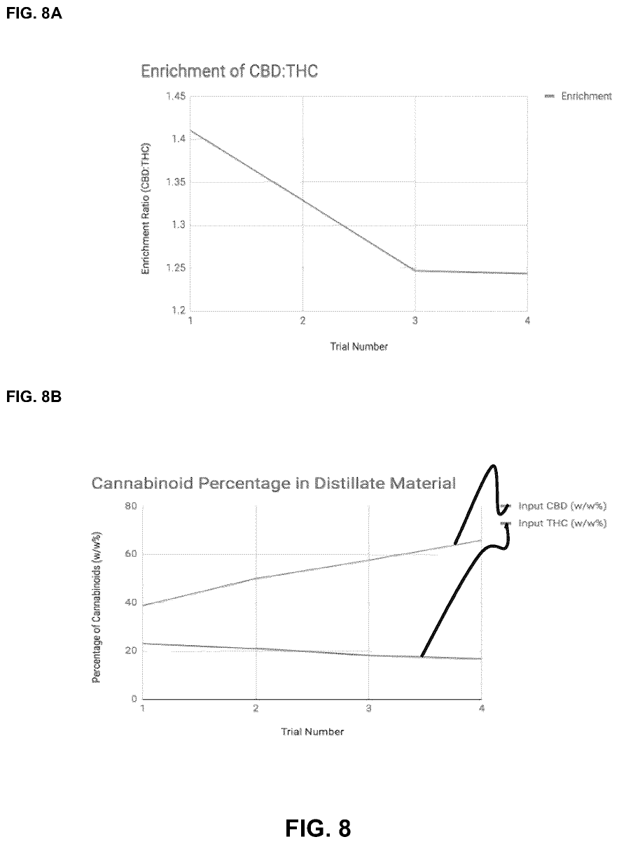

FIG. 8A shows a line graph that represents the enrichment of CBD:THC over the four trials. FIG. 8B shows a line graph that represents the percentage of CBD (upper line) and THC (lower line) in the distillate product.

Without being bound by any particular theory, these results indicate that CBD can be enriched in the distillate product at constant temperature and pressure.

### Example 8

### Variable Feed Rates

An input mixture was subjected to similar experimental conditions as described above in Example 6, but the distillation process was conducted at a constant temperature of 209° C. at a feed rate of between about 2.5 Hz and about 10 Hz and at pressure of between about 8 mmHg and 10 mmHg. Table 4 summarizes the residue/distillate accumulation over the various temperatures utilized.

TABLE 4
Residue and Distillate Accumulation Data

| | Residue/Distillate Accumulation | | | | |
|---|---|---|---|---|---|
| | | | | | Distillate/ |
| Pressure | Input Net | Residue | Distillate | Output Net | Residue |
| (mmHg) | Weight (g) | Weight (g) | Weight (g) | Weight (g) | Split |
| 2 | 213.9 | 149.3 | 67.1 | 216.4 | 0.45 |
| 8 | 610.3 | 411.4 | 194.2 | 605.6 | 0.47 |
| 10 | 596.5 | 379.6 | 187.1 | 566.7 | 0.49 |

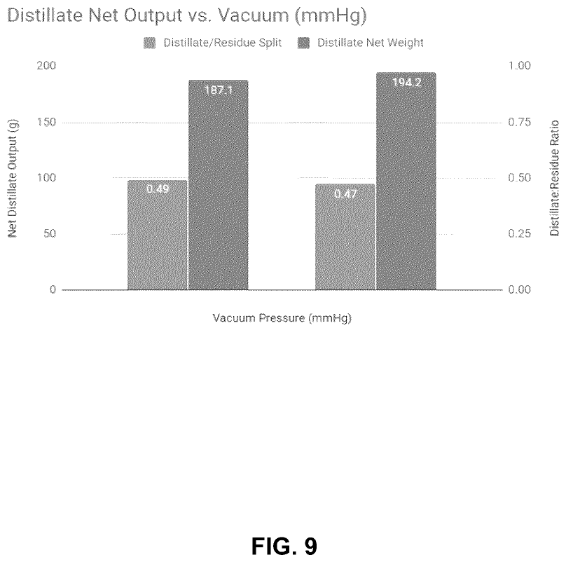

FIG. 9 shows a histogram that represents the distillate net output compared with the pressure within the distillation unit.

Without being bound by any particular theory, the ratio of distillate to residue at 210° C. with a feed rate of 10 Hz remained fairly constant even when the feed rate was decreased to 8 mmHg. Also when compared to 209° C. at 2 mmg with a feed rate of 25 Hz, the spit ratio remained substantially constant.

Table 5 shows the accumulation of residue product and distillate produce at various feed rates (2.5 Hz, 5 Hz and 10 Hz).

TABLE 5
Residue and Distillate Accumulation Data

| | Residue/Distillate Accumulation | | | | |
|---|---|---|---|---|---|
| | | | | | Distillate/ |
| Feed Rate | Input Net | Residue | Distillate | Output Net | Residue |
| (Hz) | Weight (g) | Weight (g) | Weight (g) | Weight (g) | Split |
| 10 | 676.2 | 589.3 | 59.0 | 648.3 | 0.10 |
| 5.0 | 610.3 | 411.4 | 194.2 | 605.6 | 0.47 |
| 2.5 | 314.5 | 28.9 | 276.1 | 305.0 | 9.55 |

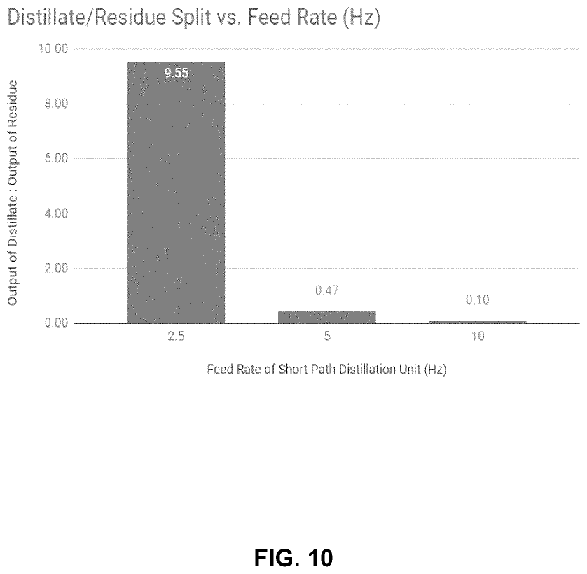

FIG. 10 shows a histogram that represents the distillate/residue split as compared to the feed rate. When using a substantially constant temperature of about 210° C. and a vacuum pressure of about 10 mmHg (13.2-13.4 mbar) for the Short Path Distillation of a Second pass resin, it was found that the most efficient feed speed to promote the greatest evaporation and distillation is about 2.5 Hz. At 2.5 Hz, the material distills over at a distillate to residue ratio of 9.55. Without being bound by any particular theory, this data demonstrates that there may be a significant time to allow for evaporation and distillation of the material. Further, the other parameters (210° C. and 10 mmHg) still promote evaporation within the evaporation chamber without degrading the cannabinoids.

### Example 9

### Omega 3 Oil

Instead of the input mixture, a mixture of omega 3 oils was subjected to a similar set of experimental conditions as Example 6. There were variable temperatures, a substantially constant pressure and a feed rate of about 89 mL/hr. Table 6 shows the accumulation of residue product and distillate product at various temperatures.

TABLE 6
Residue and Distillate Accumulation Data

| | Residue/Distillate Accumulation | | | | | |
|---|---|---|---|---|---|---|
| | | | | | Distillate/ | Percent |
| Temperature | Input Net | Residue | Distillate | Output Net | Residue | Distillate |
| (° C.) | Weight (g) | Weight (g) | Weight (g) | Weight (g) | Split | (%) |
| 160 | 136 | 83 | 22.2 | 105.2 | 0.27 | 21.1 |
| 165 | 74.7 | 38.6 | 32.4 | 71.0 | 0.84 | 45.6 |
| 170 | 76.1 | 28.6 | 46.3 | 74.9 | 1.62 | 61.8 |
| 175 | 44.4 | 3.3 | 38.2 | 41.6 | 11.61 | 93.3 |

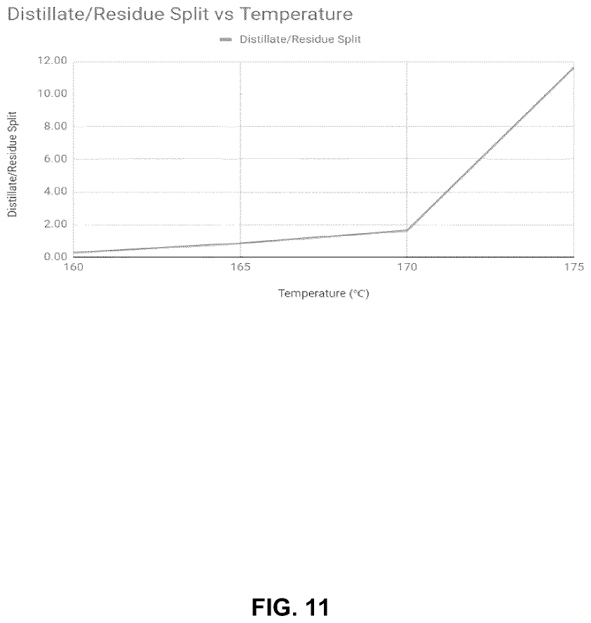

FIG. 11 shows a line graph that represents the distillate/residue split as compared to the various temperatures tested. It was found that at a temperature of about 175° C., over 93% of the Omega-3 oil INPUT was distilled. A temperature of about 175° C., at a feed speed of about 2.5 Hz and pressure of 10 mmHg produced a distillate to residue ratio of 11.61.

In the present disclosure, all terms referred to in singular form are meant to encompass plural forms of the same. Likewise, all terms referred to in plural form are meant to encompass singular forms of the same. Unless defined otherwise, all technical and scientific terms used herein have the same meaning as commonly understood by one of ordinary skill in the art to which this disclosure pertains.

As used herein, the term “about” refers to an approximately +/−10% variation from a given value. It is to be understood that such a variation is always included in any given value provided herein, whether or not it is specifically referred to.

It should be understood that the compositions and methods are described in terms of “comprising,” “containing,” or “including” various components or steps, the compositions and methods can also “consist essentially of or “consist of the various components and steps. Moreover, the indefinite articles “a” or “an,” as used in the claims, are defined herein to mean one or more than one of the element that it introduces.

For the sake of brevity, only certain ranges are explicitly disclosed herein. However, ranges from any lower limit may be combined with any upper limit to recite a range not explicitly recited, as well as, ranges from any lower limit may be combined with any other lower limit to recite a range not explicitly recited, in the same way, ranges from any upper limit may be combined with any other upper limit to recite a range not explicitly recited. Additionally, whenever a numerical range with a lower limit and an upper limit is disclosed, any number and any included range falling within the range are specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values even if not explicitly recited. Thus, every point or individual value may serve as its own lower or upper limit combined with any other point or individual value or any other lower or upper limit, to recite a range not explicitly recited.

Therefore, the present disclosure is well adapted to attain the ends and advantages mentioned as well as those that are inherent therein. The particular embodiments disclosed above are illustrative only, as the present disclosure may be modified and practiced in different but equivalent manners apparent to those skilled in the art having the benefit of the teachings herein. Although individual embodiments are discussed, the disclosure covers all combinations of all those embodiments. Furthermore, no limitations are intended to the details of construction or design herein shown, other than as described in the claims below. Also, the terms in the claims have their plain, ordinary meaning unless otherwise explicitly and clearly defined by the patentee. It is therefore evident that the particular illustrative embodiments disclosed above may be altered or modified and all such variations are considered within the scope and spirit of the present disclosure. If there is any conflict in the usages of a word or term in this specification and one or more patent(s) or other documents that may be incorporated herein by reference, the definitions that are consistent with this specification should be adopted.

Many obvious variations of the embodiments set out herein will suggest themselves to those skilled in the art in light of the present disclosure. Such obvious variations are within the full intended scope of the appended claims.

1. An apparatus for isolating one or more cannabinoids from an input mixture, the apparatus comprising:
a. a first reaction vessel that is configured to receive the input mixture and carry out a first derivatization reaction to provide a derivatized input mixture that comprises one or more derivatized cannabinoids;
b. a volatizing unit that is configured to receive and volatilize the derivatized input mixture into a derivatized cannabinoid-containing vapor stream and a residue; and
c. a distillation unit configured to receive the derivatized cannabinoid-containing vapor stream and to separate a first derivatized cannabinoid within the derivatized cannabinoid-containing vapor stream from at least a second cannabinoid.

2. The apparatus of claim 1, further comprising a second reaction vessel that is configured to receive the first derivatized cannabinoid and to carry out at least one chemical reaction that reverses the first derivatization reaction.

3. The apparatus of claim 1, wherein the volatizing unit is an evaporator, a wiped film evaporator, a short path distillation unit, a rising-falling film evaporator, a pot still, a jacketed or heated vessel, a centrifugal evaporator, a centrifugal short path distillation or a combination thereof.

4. The apparatus of claim 1, wherein the distillation unit is a fractional distillation unit.

5. (canceled)

6. (canceled)

7. The apparatus of claim 1, wherein the distillation unit defines a plenum that is configured to separate the first derivatized cannabinoid from the second cannabinoid and optionally any one or more additional cannabinoids.

8. (canceled)

9. (canceled)

10. The apparatus of claim 1, wherein the first reaction vessel is further configured to carry out a second derivatization reaction to provide a second derivatized cannabinoid.

11. The apparatus of claim 10, wherein the distillation unit is configured to separate the second derivatized cannabinoid from at least the second cannabinoid.

12. The apparatus of claim 1, wherein the derivatized cannabinoid is a mono-alkyl ester cannabinoid; a di-alkyl ester cannabinoid; a mono-aryl ester cannabinoid; a di-aryl ester cannabinoid; a monosulfonate cannabinoid; a disulfonate cannabinoid; a monosulfonic acid ester cannabinoid; a disulfonic acid ester cannabinoid; a mono-alkyl ether cannabinoid; a di-alkyl ether cannabinoid; a mono-alkyl silyl ether cannabinoid; a di-alkyl silyl ether cannabinoid; or a combination thereof.

13. The apparatus of claim 1, wherein the input mixture is a cannabis resin.

14. A method of isolating one or more cannabinoids from an input mixture, the method comprising steps of:
a. derivatizing one or more cannabinoids in the input mixture to form a derivatized input mixture that comprises one or more derivatized cannabinoids;
b. volatilizing the derivatized input mixture to provide a derivatized cannabinoid-containing vapor stream and a residue;
c. conducting the derivatized cannabinoid-containing vapor stream to a distillation unit to separate a first derivatized cannabinoid within the derivatized cannabinoid-containing vapor stream from at least a second cannabinoid; and
d. collecting a product that comprises the first derivatized cannabinoid.

15. The method according to claim 14, further comprising a step of performing at least one chemical reaction on the product to reverse a first derivatization and provide one or more isolated cannabinoids.

16. The method according to claim 14, wherein the volatizing is performed using an evaporator, a wiped film evaporator, a short path distillation unit, a rising-falling film evaporator, a pot still, a jacketed or heated vessel, a centrifugal evaporator, a centrifugal short path distillation or any combination thereof.

17. The method according to claim 14, wherein the distillation unit is a fractional distillation unit.

18. The method according to claim 1, wherein the step of conducting the derivatized cannabinoid-containing vapor stream to the distillation unit further separates the first derivatized cannabinoid from the second cannabinoid and any one or more additional cannabinoids.

19. (canceled)

20. The method according to claim 14, wherein the step of conducting the derivatized cannabinoid-containing vapor stream to the distillation unit further separates a second derivatized cannabinoid from at least the second cannabinoid.

21. The method according to claim 20, further comprising a step of performing at least one chemical reaction on the product to reverse a second derivatization and provide one or more isolated cannabinoids.

22. The method according to claim 14, wherein the derivatized cannabinoid is a mono-alkyl ester cannabinoid; a di-alkyl ester cannabinoid; a mono-aryl ester cannabinoid; a di-aryl ester cannabinoid; a monosulfonate cannabinoid; a disulfonate cannabinoid; a monosulfonic acid ester cannabinoid; a disulfonic acid ester cannabinoid; a mono-alkyl ether cannabinoid; a di-alkyl ether cannabinoid; a mono-alkyl silyl ether cannabinoid; a di-alkyl silyl ether cannabinoid; or a combination thereof.

23. The method according to claim 14, wherein the derivatized cannabinoid is a mono-alkyl ester cannabidiol (CBD); a mono-alkyl ester cannabigerol (CBG); a mono-alkyl ester tetrahydrocannabinol (THC); a mono-alkyl ester cannabielsoin (CBE); a mono-alkyl ester cannabinol (CBN); a mono-alkyl ester cannabicyclol (CBL); a mono-alkyl ester tetrahydrocannabivarin (THCV); a mono-alkyl ester cannabidivarin (CBDV); a di-alkyl ester CBD; a di-alkyl ester CBG; a di-alkyl ester THC; a di-alkyl ester CBE;
a di-alkyl ester CBN; a di-alkyl ester CBL; a di-alkyl ester THCV; a di-alkyl ester CBDV; a mono-aryl ester CBD; a mono-aryl ester CBG; a mono-aryl ester THC; a mono-aryl ester CBE; a mono-aryl ester CBN; a mono-aryl ester CBL; a mono-aryl ester THCV; a mono-aryl ester CBDV; a di-aryl ester CBD; a di-aryl ester CBG; a di-aryl ester THC; a di-aryl ester CBE; a di-aryl ester CBN; a di-aryl ester CBL; a di-aryl ester THCV; a di-aryl ester CBDV; a monosulfonate CBD; a monosulfonate CBG; a monosulfonate THC; a monosulfonate CBE; a monosulfonate CBN; a monosulfonate CBL; a monosulfonate THCV; a monosulfonate CBDV; a disulfonate CBD; a disulfonate CBG; disulfonate THC; a disulfonate CBE; a disulfonate CBN; a disulfonate CBL; a disulfonate THCV; a disulfonate CBDV; a monosulfonic acid ester CBD; a monosulfonic acid ester CBG; a monosulfonic acid ester THC; a monosulfonic acid ester CBE; a monosulfonic acid ester CBN; a monosulfonic acid ester CBL; a monosulfonic acid ester THCV;
a monosulfonic acid ester CBDV; a disulfonic acid ester CBD; a disulfonic acid ester CBG; a disulfonic acid ester THC; a disulfonic acid ester CBE; a disulfonic acid ester CBN; a disulfonic acid ester CBL; a disulfonic acid ester THCV; a disulfonic acid ester CBDV; a mono-alkyl ether CBD; a mono-alkyl ether CBG; a mono-alkyl ether THC; a mono-alkyl ether CBE; a mono-alkyl ether CBN; a mono-alkyl ether CBL; a mono-alkyl ether THCV; a mono-alkyl ether CBDV; a di-alkyl ether CBD; a di-alkyl ether CBG; a di-alkyl ether THC; a di-alkyl ether CBE; a di-alkyl ether CBN; a di-alkyl ether CBL; a di-alkyl ether THCV; a di-alkyl ether CBDV; a mono-alkyl silyl ether CBD; a mono-alkyl silyl ether CBG; a mono-alkyl silyl ether THC; a mono-alkyl silyl ether CBE; a mono-alkyl silyl ether CBN; a mono-alkyl silyl ether CBL; a mono-alkyl silyl ether THCV; a mono-alkyl silyl ether CBDV; a di-alkyl silyl ether CBD; a di-alkyl silyl ether CBG; a di-alkyl silyl ether THC; a di-alkyl silyl ether CBE; a di-alkyl silyl ether CBN; a di-alkyl silyl ether CBL; a di-alkyl silyl ether THCV; or a di-alkyl silyl ether CBDV.

24. The method according to claim 14, wherein the derivatized cannabinoid is a mono-alkyl ester cannabinoid, a di-alkyl ester cannabinoid, a mono-aryl ester cannabinoid, a di-aryl ester cannabinoid, a monosulfonate ester cannabinoid, a disulfonate ester cannabinoid, a monosulfonic acid ester cannabinoid, a disulfonic acid ester cannabinoid, a mono-alkyl ether cannabinoid, a di-alkyl ether cannabinoid, a monosilyl ether cannabinoid, or a disilyl ether cannabinoid, and the isolated cannabinoid is THC, Δ8-THC, trans-Δ10-THC, cis-Δ10-THC, THCV, Δ8-THCV, Δ49-THCV, CBD, CBDV, CBC, CBCV, CBG, CBGV, CBN, CBNV, CBND, CBNDV, CBE, CBEV, CBL, CBLV, CBT, cannabicitran, or a combination thereof.

25.-35. (canceled)

36. The method according to claim 15, wherein the at least one chemical reaction converts the derivatized cannabinoid from a mono-alkyl ester cannabinoid a di-alkyl ester cannabinoid, a mono-aryl ester cannabinoid, a di-aryl ester cannabinoid, a monosulfonate ester cannabinoid, a disulfonate ester cannabinoid, a monosulfonic acid ester cannabinoid, a disulfonic acid ester cannabinoid, a mono-alkyl ether cannabinoid, a di-alkyl ether cannabinoid, a monosilyl ether cannabinoid, or a disilyl ether cannabinoid to THC, Δ8-THC, trans-Δ10-THC, cis-Δ10-THC, THCV, Δ8-THCV, Δ9-THCV, CBD, CBDV, CBC, CBCV, CBG, CBGV, CBN, CBNV, CBND, CBNDV, CBE, CBEV, CBL, CBLV, CBT, or cannabicitran.

37.-47. (canceled)

48. The method according to claim 14, wherein the input mixture is a cannabis resin.

49. A method of isolating one or more cannabinoids from an input mixture, the method comprising steps of:
a. volatilizing the input mixture to provide a cannabinoid-containing vapor stream and a residue;
b. derivatizing one or more cannabinoids in the cannabinoid-containing vapor stream to form a derivatized cannabinoid-containing vapor stream that comprises one or more derivatized cannabinoids;
c. conducting the derivatized cannabinoid-containing vapor stream to a distillation unit to separate a first derivatized cannabinoid within the derivatized cannabinoid-containing vapor stream from at least a second cannabinoid;
d. and collecting a product that comprises the first derivatized cannabinoid.
